SEQUENCE LISTING (63 sequences)

SEQ ID NO: 1 - DNA, 27 bp, Artificial Sequence
  Linker sequence
gggcccggctcggacgaagtggatatc

SEQ ID NO: 2 - protein, 8 aa, Artificial Sequence
  Linker sequence
GPGSDEVD

SEQ ID NO: 3 - DNA, 48 bp, Artificial Sequence
  Linker sequence
gggcccggctcggggggaggcggttcaggaggtgacgaagtggatatc

SEQ ID NO: 4 - protein, 15 aa, Artificial Sequence
  Linker sequence
GPGSGGGGSGGDEVD

SEQ ID NO: 5 - DNA, 48 bp, Artificial Sequence
  Linker sequence
gggcccggctcgcatcatcaccatcaccatggagacgaagtggatatc

SEQ ID NO: 6 - protein, 15 aa, Artificial Sequence
  Linker sequence
GPGSHHHHHHGDEVD

SEQ ID NO: 7 - DNA, 48 bp, Artificial Sequence
  Linker sequence
gggcccggctcgcatcaccaccataaggacgatgacgaagtggatatc

SEQ ID NO: 8 - protein, 15 aa, Artificial Sequence
  Linker sequence
GPGSHHHHKDDDEVD

SEQ ID NO: 9 - DNA, 48 bp, Artificial Sequence
  Linker sequence
gggcccgatgacggcgaaggagagaaggacgatgacgaagtggatatc

SEQ ID NO: 10 - protein, 15 aa, Artificial Sequence
  Linker sequence
GPDDGEGEKDDDEVD

SEQ ID NO: 11 - protein, 4 aa, Artificial Sequence
  Caspase target sequence
WEHD

SEQ ID NO: 12 - protein, 4 aa, Artificial Sequence
  Caspase target sequence
LEHD

SEQ ID NO: 13 - protein, 4 aa, Artificial Sequence
  Caspase target sequence
DEVD

SEQ ID NO: 14 - protein, 4 aa, Artificial Sequence
  Caspase target sequence
DEHD

SEQ ID NO: 15 - protein, 4 aa, Artificial Sequence
  Caspase target sequence
VEHD

SEQ ID NO: 16 - protein, 4 aa, Artificial Sequence
  Caspase target sequence
LEHD

SEQ ID NO: 17 - protein, 5 aa, Artificial Sequence
  Caspase target sequence
DEVDS

SEQ ID NO: 18 - protein, 4 aa, Artificial Sequence
  Caspase target sequence
DETD

SEQ ID NO: 19 - protein, 4 aa, Artificial Sequence
  Caspase target sequence
DEMD

SEQ ID NO: 20 - protein, 4 aa, Artificial Sequence
  Caspase target sequence
DELD

SEQ ID NO: 21 - protein, 4 aa, Artificial Sequence
  Caspase target sequence
DEPD

SEQ ID NO: 22 - protein, 4 aa, Artificial Sequence
  Caspase target sequence
DEAD

SEQ ID NO: 23 - protein, 4 aa, Artificial Sequence
  Caspase target sequence
DEQD

SEQ ID NO: 24 - protein, 4 aa, Artificial Sequence
  Caspase target sequence
DTVD

SEQ ID NO: 25 - protein, 4 aa, Artificial Sequence
  Caspase target sequence
DQTD

SEQ ID NO: 26 - protein, 4 aa, Artificial Sequence
  Caspase target sequence
DITD

SEQ ID NO: 27 - protein, 4 aa, Artificial Sequence
  Caspase target sequence
DMQD

SEQ ID NO: 28 - protein, 4 aa, Artificial Sequence
  Caspase target sequence
DSLD

SEQ ID NO: 29 - protein, 4 aa, Artificial Sequence
  Caspase target sequence
DGPD

SEQ ID NO: 30 - protein, 4 aa, Artificial Sequence
  Caspase target sequence
DVPD

SEQ ID NO: 31 - protein, 4 aa, Artificial Sequence
  Caspase target sequence
DRHD

SEQ ID NO: 32 - protein, 4 aa, Artificial Sequence
  Caspase target sequence
DHVD

SEQ ID NO: 33 - protein, 4 aa, Artificial Sequence
  Caspase target sequence
DPSD

SEQ ID NO: 34 - protein, 4 aa, Artificial Sequence
  Caspase target sequence
DSVD

SEQ ID NO: 35 - protein, 4 aa, Artificial Sequence
  Caspase target sequence
DSLD

SEQ ID NO: 36 - protein, 4 aa, Artificial Sequence
  Caspase target sequence
DSYD

SEQ ID NO: 37 - protein, 4 aa, Artificial Sequence
  Caspase target sequence
DQTD

SEQ ID NO: 38 - protein, 4 aa, Artificial Sequence
  Caspase target sequence
DDVD

SEQ ID NO: 39 - protein, 4 aa, Artificial Sequence
  Caspase target sequence
DAGD

SEQ ID NO: 40 - protein, 4 aa, Artificial Sequence
  Caspase target sequence
MELD

SEQ ID NO: 41 - protein, 4 aa, Artificial Sequence
  Caspase target sequence
VEVD

SEQ ID NO: 42 - protein, 4 aa, Artificial Sequence
  Caspase target sequence
LEVD

SEQ ID NO: 43 - protein, 4 aa, Artificial Sequence
  Caspase target sequence
LQTD

SEQ ID NO: 44 - protein, 4 aa, Artificial Sequence
  Caspase target sequence
VSWD

SEQ ID NO: 45 - protein, 4 aa, Artificial Sequence
  Caspase target sequence
YVPD

SEQ ID NO: 46 - protein, 4 aa, Artificial Sequence
  Caspase target sequence
SHVD

SEQ ID NO: 47 - protein, 4 aa, Artificial Sequence
  Caspase target sequence
HLAD

SEQ ID NO: 48 - protein, 4 aa, Artificial Sequence
  Caspase target sequence
ARQD

SEQ ID NO: 49 - protein, 4 aa, Artificial Sequence
  Caspase target sequence
PAPD

SEQ ID NO: 50 - protein, 4 aa, Artificial Sequence
  Caspase target sequence
SSTD

SEQ ID NO: 51 - protein, 4 aa, Artificial Sequence
  Caspase target sequence
LGTD

SEQ ID NO: 52 - protein, 4 aa, Artificial Sequence
  Caspase target sequence
LSVD

SEQ ID NO: 53 - protein, 4 aa, Artificial Sequence
  Caspase target sequence
ALDD

SEQ ID NO: 54 - protein, 4 aa, Artificial Sequence
  Caspase target sequence
LSSD

SEQ ID NO: 55 - protein, 4 aa, Artificial Sequence
  Caspase target sequence
ALAD

SEQ ID NO: 56 - protein, 4 aa, Artificial Sequence
  Caspase target sequence
FIQD

SEQ ID NO: 57 - protein, 4 aa, Artificial Sequence
  Caspase target sequence
DDDK

SEQ ID NO: 58 - protein, 4 aa, Artificial Sequence
  Caspase target sequence
RGPR

SEQ ID NO: 59 - protein, 183 aa, Mus musculus
MGSPLPITPVNATCAIRHPCHGNLMNQIKNQLAQLNGSANALFISYYTAQGEPFPNNVEKLCAPNMTDFPSFHGNGTEKTKLVELYRMVAYLSASLTNITRKDQKVLNPTAVSLQVKLNATIDVMRGLLSNVLCRLCNKYRVGHVDVPPVPDHSDKEAFQRKKLGCQLLGTYKQVISVVVQAF

SEQ ID NO: 60 - protein, 165 aa, Homo sapiens
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDILAVRKYFQRITLYLKEKKYSPCAWEVVRAEIMRSFSLSTNLQESLRSKE

SEQ ID NO: 61 - protein, 220 aa, mammalian
SPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDL

SEQ ID NO: 62 - protein, 111 aa, Escherichia coli
SDKIIHLTDDSFDTDVLKADGAILVDFWAEWCGPCKMIAPILDEIADEYQGKLTVAKLNIDQNPGTAPKYGIRGGIPTLLLFKNGEVAATKVGALSKGQLKEFLDANLAGS

SEQ ID NO: 63 - protein, 4 aa, Artificial Sequence
  Caspase target sequence
DXXD

(19) United States
(12) Patent Application Publication
(10) Pub. No.: US 2006/0099689 A1
(43) Pub. Date: May 11, 2006

(54) Use of caspase enzymes for maturation of engineered recombinant polypeptide fusions

(76) Inventors: Nico M. A. C. Mertens, Melsele (BE); Andrew Graham Kelly, Carcavelos (PT)

Correspondence Address: KNOBBE MARTENS OLSON & BEAR LLP, 2040 MAIN STREET, FOURTEENTH FLOOR, IRVINE, CA, 92614, US

(21) Appl. No.: 10/543,587

(22) PCT Filed: Feb. 23, 2004
(86) PCT No.: PCT/EP04/01759

§ 371 Date: Sep. 2, 2005

(30) Foreign Application Priority Data
Feb. 21, 2003 (EP) 03447034.4

(51) Int. Cl. C12P 21/04; C12N 9/64; C12N 15/74; C12N 1/21

(52) U.S. Cl. 435/69.7; 435/226; 435/320.1; 435/252.33

(57) ABSTRACT

The invention relates to methods of producing and isolating recombinant proteins using fusion protein constructs comprising specific recognition sites for a maturating protease. The invention specifically relates to the use of caspase proteases for the maturation of engineered recombinant fusion proteins or polypeptides. These molecules are engineered using recombinant DNA technology to comprise a specific target sequence for a caspase between a first fusion part and the polypeptide of interest. After processing, the desired mature format of the protein or polypeptide is obtained.

### FIELD OF THE INVENTION

The invention relates generally to the field of molecular biology, biotechnology or process engineering for the production of proteins or peptides. The invention relates to methods of producing and isolating recombinant proteins using fusion protein constructs comprising specific recognition sites for a maturating protease. The invention specifically relates to the use of caspase proteases for the maturation of engineered recombinant fusion proteins or polypeptides. These molecules are engineered using recombinant DNA technology to comprise a specific target sequence for a caspase between a first fusion part and the polypeptide of interest. After processing, the desired mature format of the protein or polypeptide is obtained.

### BACKGROUND OF THE INVENTION

The heterologous expression of recombinant proteins is hampered by several technological difficulties. The expression level can be compromised due to a variety of causes.

First, the level of translation can be hampering a reasonable protein production level. This needs to be engineered and optimized for each individual protein, which is a time-consuming task. Second, the protein of interest can be unstable. This instability can be due to several causes. It has been described that the N-terminus of a protein is determining the overall stability of the protein, which will determine its likelihood of being degraded by host proteases. Third, despite high yield of expression of recombinant proteins, the major setback of producing recombinant proteins in E. coli is the formation of insoluble precipitates of the expressed protein. These “inclusion bodies” are in most cases biologically inactive. Therefore, a refolding process is required to regain soluble protein that is biologically active. This is a cumbersome process, and implementation in a production environment is raising the cost of the process considerably. The refolding process is by no means a guarantee of full biological activity of the protein. Isomers can be formed with the same biophysical characteristics, but with a much lower biological activity. Usually, these folding isomers have a different hydrophobic surface and can be identified by reverse phase chromatography. Furthermore, when producing mature proteins in the cytoplasm of E. coli, the removal of the obligatory N-terminal methionine amino acid might not be as efficient, resulting in a non-controlled heterogenicity in the protein preparation. The N-terminal methionine is also formylated in E. coli, which is unwanted as it is a specific immunogenic signal in human and animal.

These problems can be overcome by adapting a fusion protein strategy. Fusion proteins are extensions of the protein of interest with one or more amino acids. Fusion proteins can be constructed in order to couple a peptide tag to the protein of interest, or to create a fusion with a larger polypeptide. A preferred strategy is to fuse the additional polypeptide part at the N-terminus of the polypeptide of interest. Advantages of using this fusion protein strategy include the fact that with a proper fusion partner, expression is generally high and need no further optimization, as the N-terminus can be chosen to stabilize the fusion protein from degradation, whereby the fusion protein is generally better protected against protease degradation, and solubility of the fusion construct is often improved. Furthermore, a specific simple purification method can be designed targeting the fusion part, and allowing the fusion protein to be isolated in a simple chromatographic step. If such a purification step does not exists for the fusion part, affinity “tags” can be fused to the fusion part. The newly composed fusion part then has acquired specific characteristic behaviors on an adsorption column. Examples are peptide tags recognized by specific antibodies, peptide tags with an affinity towards a certain protein, protein domains with an affinity towards another protein or protein domain, specifically isolated binding peptides, protein domains or proteins, and proteins or domains thereof that specifically bind a non-pertinacious substrate. One or more of these affinity tags may be combined in the fusion protein.

For this purpose fusion partners have been identified empirically, on the basis on how well they are produced in E. coli, or on the basis of their hydrophilicity. Examples of empirically identified partners increasing the soluble expression of the protein fused to the polypeptide of interest are GST, thioredoxin or MBP. Examples of fusion partners chosen on the basis of their high hydrophilic character are NusA and GrpE. The result, however, is still unprecictable, depending on the intrinsic properties of the protein that is expressed. Examples of the affinity tags mentioned are poly-histidine tags, poly-arginine tags, peptide substrates for antibodies, chitin binding domain, the RNase S peptide, maltose binding protein, gluthathion S-transferase, protein A, beta-galactosidase.

Although interesting, the fusion protein approach shifts the bottleneck of the production process to a different level:—the removal of the fusion part and maturation of the protein of interest.

In order to obtain a mature protein of interest starting from the fusion protein, the fusion partner or the composed fusion part must be removed from the protein of interest. To this end, several methods have been described. Chemical cleavage methods have been devised for cleavage after specific amino acids. However, the amino acid preceding the first amino acid of the mature part of the protein of interest should then be unique, or at least not occur in the protein of interest. Also, the harsh chemical conditions needed for the induction of this cleavage can lead to chemical protein degradation: oxidation, deamidation, cyclic imide formation, iso-aspartate formation are just examples of this. Chemical protein degradation will lead to heterogeneity in the final protein preparation; it may introduce antigenic determinants in the protein or diminish its activity, making it less suitable for use as a therapeutic substance. Proteases have been described that have some specificity towards certain peptide sequences. In order to use these proteases a linker is introduced between the fusion part and the protein of interest, to compose a fusion protein of the formula
FᵢL-P
Where F represents the fusion part that can be composed of different units F₁, F₂, . . . Fₙ, L is a linker sequence that contains a recognition sequence for the protease used and possibly also a spacer to increase the chance of the protease site to be accessible and P is the protein of interest.

Some of the proteases used for this purpose are cutting within the linker, as their specificity is determined both by the upstream sequence of the cleavage site (Pᵢ positions, numbered away from the cleavage site towards the N-terminus) and by the downstream sequence of the cleavage site (P′ᵢ positions, numbered away from the cleavage site towards the C-terminus). Processing with these enzymes will not result in obtaining a mature protein, but in a protein still fused to a number of non-native amino acids. While for a number of biochemical purposes this might be well acceptable, in the development of a therapeutic reagent it is highly desirable to obtain but the mature sequence of the desired protein of peptide of interest. Examples of such proteases and examples of the cutting sequences are:

| ENZYME | SEQUENCE |
|---|---|
| IgA protease | Thr-Pro-Ala-Pro-Arg-Pro-Pro\|\hat{ }\|Thr-Pro |
| Collagenase | Pro-Xaa\|\hat{ }\|Gly-Pro |
| HRV3C | Leu-Glu-Val-Leu-Phe-Gln\|\hat{ }\|Gly-Pro |
| TEV | Glu-Xaa-Xaa-Tyr-Gln\|\hat{ }\|(Gly/Ser) |

The notation ‘|\hat{ }|’ refers to the position in the sequence where the protease cuts.

Other enzymes show no or very little preference for the P′₁ and P′₂ position, and thus allow maturation of the fusion protein to obtain the protein of interest, without any artificial amino acids fused to it. Obviously, for obtaining material for therapeutic use in human and animal, this is the most preferred option.

Examples of such restricted proteases and an example of the cutting sequence with no specificity C-terminal to the cleavage point are:

| ENZYME | SEQUENCE |
|---|---|
| Trypsin | Arg\|\hat{ }\|, Lys\|\hat{ }\| |
| Thrombin | Arg-Gly-Pro-Arg\|\hat{ }\| |
| Factor Xa | Ile-Glu-Gly-Arg\|\hat{ }\| |
| Enterokinase | Asp-Asp-Asp-Lys\|\hat{ }\| |

Enzymes like Trypsin obviously have only a very limited use, since there is a high chance that they also degrade the protein of interest.

The specificity of Thrombin is also low. Optimum cleavage sites for Thrombin are given below. In the first three examples, the formula for the recognition site is

P4-P3-P-R/K|ˆ|P1′-P2′

where P3 and P4 are hydrophobic amino acids and P1′ and P2′ are non-acidic amino acids. The bond behind R/K is cleaved. Possible cleavage sites of thrombin are however variable (one letter code for amino acids is shown):

| | | | | | |
|---|---|---|---|---|---|
| P4 | P3 | P2 | P1 | P1′ | P2′ |

| | | | | | | |
|---|---|---|---|---|---|---|
| 1 | M | Y | P | R | G | N |
| 2 | I | R | P | K | L | K |
| 3 | L | V | P | R | G | S |
| 4 | | | A | R | G | |
| 5 | | | G | K | A | |

In the cases 4 and 5, shown above, the cleavage site is less defined and sites similar to these sequences will be regularly found in natural proteins. Thrombin therefore seems to be only little more specific than Trypsin. For this reason, Thrombin cleavage will also have a high change of hydrolysing the protein of interest. The specificity of Factor Xa is less stringent and examples are known where the protein of interest is cleaved at unsuspected sites, and specificity is not higher than the one observed with Thrombin. This often leads to processing and degradation of the protein of interest. Further degradation of the clipped protein parts is often due to activity of contaminating proteases. Moreover, the production of Factor Xa is rather difficult, since it needs to be produced in a mammalian cell-based expression system and it requires post-productional activation steps (activation by Russell viper venom). This makes the enzyme not the best choice for large-scale production since cost of enzyme might outrun the cost of production of the fusion protein comprising the protein of interest.

Apart from the lack of specificity of these proteases, the efficiency of processing and the yield of mature protein obtained is rather low, and often insufficient to consider the development of a production process using these enzymes.

Bovine Enterokinase is an enzyme with a higher degree of specificity, but several cases are known where the enzyme did not cleave the protein of interest or degraded the protein of interest in an unexpected manner. Moreover, the Enterokinase or its catalytic domain is difficult to produce and purify.

### SUMMARY OF THE INVENTION

The present invention uses a process to obtain mature protein, a protein domain or a peptide starting with any amino acid of choice at its N-terminus by producing it as a fusion protein to an N-terminal fusion partner via connection with an engineered linker sequence and thereafter releasing it in a specific way by incubation with a protease that belongs to the caspase family of cysteine proteases. The invention also relates to the design of peptide linkers used to connect a fusion partner to the mature protein whereby said linker is specifically digested by such a caspase. The invention further relates to the expression and purification of fusion proteins comprising a fusion part and a mature protein or peptide of interest comprising a linker sequence designed to be processed by a caspase protease. This invention can be used to facilitate production of a recombinant protein in a functional and/or mature form and to facilitate recovery processes.

This invention describes the use of caspases as maturation proteases for recombinant fusion proteins containing an engineered caspase recognition site in the linker connecting the fusion protein and the protein of interest. The present invention describes a process using caspases to separate the fusion part and the linker sequence from a protein of interest without limitation of choice of the first amino acid of the mature protein, making the method suitable for producing human and animal therapeutics. Also, the caspase enzymes can be produced in an efficient and cheap way in an E. coli expression system and can be subsequently purified leaving no traces of host proteases. These characteristics make the invention suitable for process development of the production of peptides, proteins or part of proteins at large scale. The present invention results in a method that cleaves the fusion part and the linker sequence with high specificity, and several specificities can be engineered in the linker depending on the caspase used. Surprisingly, it was found that even proteins with suspected cleavage sites on the basis of their protein sequence were only cut at the engineered site and not at cryptic internal sites. Furthermore, cleavage times can be as short as several minutes to one hour. The enzyme can be specifically removed from the processing reaction and several protease inhibitors are known to the person skilled in the art, which allow to end the processing reaction in a controlled way.

In one embodiment, the present invention relates to a method for producing a polypeptide of interest in a biologically active form, comprising:
producing, in a suitable host, preferably a prokaryote, more preferably Escherichia coli, a fusion protein comprising an aminoterminal polypeptide, a linker sequence comprising a caspase recognition site and the polypeptide of interest,
cleaving said fusion protein by a caspase which specifically cleaves the fusion protein at the junction of the linker sequence and the amino-terminal amino acid residue of the mature polypeptide of interest, and
isolating the biologically active polypeptide of interest.

In a further embodiment, the invention relates to a method as defined above wherein said caspase recognition site comprises the amino acid sequence DXXD (SEQ ID NO 63), preferably DEVD (SEQ ID NO 13) or DEHD (SEQ ID NO 14).

The invention further relates to any of the methods defined above wherein said caspase is selected from the group consisting of caspase-2, caspase-3, caspase-7, CED-3, reversed caspase-3 and reversed caspase-7.

According to a further embodiment, the invention relates to any of the methods defined above wherein the linker sequence in the fusion protein comprises at least 4, preferably at least 5, 6, 7, 8, 9, 10, 11, 12, 13, 14, 15, 16, 17, 18, 19 or 20 amino acids, more preferably at least 25, 30 or 40 amino acids; preferably said linker sequence is represented by any of SEQ ID NOs 2, 4, 6, 8 or 10.

According to a preferred embodiment of the methods of the invention, the linker sequence of the fusion protein consists of the amino acid sequence DEVD or DEHD.

The invention further relates to a method as defined above wherein the linker sequence as defined above comprises at least 2, 3, 4 or 5 hydrophilic, or at least 2, 3, 4 or 5 charged, or at least 2, 3, 4 or 5 small amino acids.

According to a further embodiment, the invention relates to a method as defined above wherein said cleaving of the fusion protein by the caspase is carried out in vitro; preferably a yield of at least 80% of mature polypeptide of interest is obtained within 60 minutes as measured or judged by densitiometric scanning.

In another embodiment, the invention relates to a method according to any of the methods described above further characterized in that the caspase protein is produced in the same host as the fusion protein; preferably, the caspase protein sequence is comprised in, i.e. is part of, the sequence of the fusion protein.

In one embodiment, in the methods of the invention, the production (and/or the induction of the production) of the caspase protein and the production (and/or the induction of the production) of the fusion protein are sequential, preferably, the caspase protein is produced (and/or the production of the caspase protein is induced) after the production (and/or the induction of the production) of the fusion protein.

The invention further relates to any of the methods described above wherein said cleaving of the fusion protein is carried out in vivo.

According to another embodiment, the invention relates to a method for producing a polypeptide of interest in a biologically active (and/or mature) form comprising the following steps:
producing in E. coli, a fusion protein comprising an aminoterminal polypeptide, a linker sequence comprising a caspase recognition site and the polypeptide of interest, wherein the linker sequence is represented by any of SEQ ID NOs 2, 4, 6, 8 or 10,
cleaving said fusion protein by a caspase which specifically cleaves the fusion protein at the junction of the linker sequence and the amino-terminal amino acid residue of the mature polypeptide of interest, and
isolating the biologically active polypeptide of interest.

According to a further embodiment the invention relates to a method for producing a polypeptide of interest in a biologically active (and/or mature) form comprising the following steps:
producing in E. coli (1) a fusion protein comprising an aminoterminal polypeptide, a linker sequence comprising a caspase recognition site, said linker sequence preferably having the amino acid sequence represented by any of SEQ ID NOs 2, 4, 6, 8 or 10, and the polypeptide of interest; and (2) a caspase protein, preferably caspase-3 or caspase-7; wherein said fusion protein and said caspase polypeptide are under the control of a different inducible promoter,
inducing expression of the fusion protein,
inducing expression of the caspase protein,
cleaving said fusion protein in vivo by the caspase protein which cleaves the fusion protein at the junction of the linker sequence and the amino-terminal amino acid residue of the mature polypeptide of interest, and
isolating the biologically active polypeptide of interest.

The invention further relates to any of the methods described above wherein a single polynucleic acid encodes the fusion protein and the caspase, or, in the alternative, wherein separate polynucleic acids encode the fusion protein and the caspase.

According to yet another embodiment, the invention relates to a polynucleic acid encoding any of the fusion proteins described above. More particularly, the invention relates to a polynucleic acid encoding a fusion protein comprising an aminoterminal polypeptide, a mature polypeptide of interest and a linker sequence between the aminioterminal polypeptide and the mature polypeptide of interest, said linker sequence comprising a caspase recognition site at the junction between the linker and the amino terminal amino acid of the mature polypeptide of interest.

The invention further relates to a polynucleic acid as described above, further encoding a caspase protein. Preferably said caspase protein is selected from the group consisting of caspase-2, caspase-3, caspase-7, CED-3, reversed caspase-3 and reversed caspase-7.

According to other embodiments of the invention, the aminoterminal polypeptide encoded by the polynucleic acids of the invention preferably is selected from the group comprising, but not limited to, glutathione S-transferase gene GST, E. coli thioredoxin TRX, NusA, chitin binding domain CBD, chloramphenicol acetyl transferase CAT, protein A (prot A), LysRS, maltose binding protein (MBP), ubiquitin, calmodulin, DsbA, DsbC and lambda gpV.

Preferably, the linker sequence of the polynucleic acid of the invention comprises at least 5, preferably at least 6, 7, 8, 9, 10, 11, 12, 13, 14, 15, 16, 17, 18, 19 or 20 amino acids, more preferably at least 25, 30 or 40 amino acids; preferably said linker sequence is represented by any of SEQ ID NOs 2, 4, 6, 8 or 10.

According to another embodiment, said linker sequence consists of 4 amino acids, comprising a caspase recognition site consisting of the amino acid sequence DXXD, preferably DEVD or DEHD.

The invention further relates to any of the polynucleic acids described above wherein the expression of said fusion protein and/or said caspase protein is under the control of a suitable promoter, preferably said promoter is an inducible promoter, and even more preferred, the fusion protein and the caspase protein are under the control of a different inducible promoter.

The invention further relates to any of the polynucleic acids described above wherein said fusion protein further comprises a detection or affinity tag, preferably said detection or affinity tag is selected from the group consisting of a polyhistidine tag, a polyarginine tag, a streptavidin binding tag, a biotinylated tag and a tag recognized by an antibody.

The invention also relates to a vector comprising any of the polynucleic acids described above, and to a host cell comprising said vector or any of said polynucleic acids.

The invention further relates to a fusion protein encoded by the nucleic acids described above, and to an antibody specifically binding to said fusion protein.

The invention further relates to the use of any of the polynucleic acids, vectors, host cells or fusion proteins described above, for the production of a polypeptide of interest in a mature and/or biologically active form.

The invention further relates to a bioreactor suitable for the production of biologically active, i.e. functional, and/or mature proteins of interest comprising:
(a) a fusion protein encoded by any of the polynucleic acids described herein, said fusion protein comprising an affinity tag having a specific affinity for a substrate, and
(b) a substrate,
   wherein the affinity tag portion of the fusion protein is bound to the substrate. Preferably the substrate is contained within an affinity column.

According to another embodiment, the invention relates to a bioreactor suitable for the production of biologically active, i.e. functional, and/or mature proteins of interest comprising:
(a) a fusion protein encoded by any of the polynucleic acids described herein, said fusion protein comprising an affinity tag having a specific affinity for a substrate, and
(b) a caspase enzyme coupled to a solid support,
   wherein the caspase enzyme specifically cleaves the caspase recognition site in the fusion protein.

The invention also relates to a method for producing a protein of interest comprising:
(a) producing a fusion protein as described abover, said fusion protein comprising an affinity tag or an aminoterminal polypeptide having a specific affinity for a substrate,
(b) introducing said fusion protein into a bioreactor comprising a caspase enzyme coupled to a solid support,
(c) incubating said fusion protein with the caspase enzyme for at least one hour, and
(d) washing the reactor and capturing the eluted fusion protein by binding to the substrate. Preferably the substrate is contained within an affinity column.

### DETAILED DESCRIPTION OF THE INVENTION

Surprisingly we found that a highly specific and highly efficient processing can be obtained by the use of caspases as specific proteases, when using a specially designed fusion protein as substrate.

Caspases are cystein proteases that specifically cleave after an aspartyl residue. The caspases gene family thus far contains at least 14 mammalian members, of which at least 11 human enzymes are known. A first classification can be made after phylogenetic analysis and divides the family in two classes: the Caspase-1 (ICE) related enzymes and the Caspase-3 (CED-3) related enzymes. A further division can be made on the basis of short (caspase-3, -6 and -7), or long (caspase-1, -2, -4, -5, and caspases-8 to 14) prodomains. Alternatively, the proteases can be subdivided on the basis of their substrate specificity. This would divide the caspases in three different groups. Group I caspases (Caspases 1, 4, 5, 13) are liberal for substitutions at the P4 position but prefer bulky hydrophobic amino acids such as Tyr or Trp. Group II caspases (Caspases 2, 3, 7) are substantially more stringent in S4, requiring a P4 Asp. The preferred cleavage motif for this group is DEXD, but many substrates of the formula DXXD have been identified. Caspase-2 has even stringency at position P5. Table I gives a list of non-limiting examples of natural substrate proteins. It can be noted that there is a high preference for small hydrophilic amino acids at the P1′ position of the natural substrates and that the consensus sequence is not met by a large group of target sequences. It should also be remembered that not all targets are cleaved with the same efficiency. Group III caspases (Caspases 6, 8, 9, 10) prefer branched chain aliphatic amino acids in P4.

Fusion protein strategies for enhancing expression level, improving solubility and facilitating purification of the protein have been around since 1983 and before. None of these strategies has been used in a large-scale production process. Obviously, the bottleneck of adapting a fusion protein strategy for large-scale process development is in the specificity, activity, availability and purity of the protease enzyme used. The specificity needs to be high enough to at least allow a number of proteins to be cleaved only at the engineered cleavage site in the connecting linker sequence. The activity of the enzyme needs to be high enough to allow sufficient cleavage in a short period of time. This will avoid hold-up time during the production and minimizes degradation of the protein of interest during incubation. The protease needs to be available at low cost, so an efficient expression system and a low-cost production method are necessary. Therefore, an engineered protease containing an affinity tag might be a good solution. The protease should also be sufficiently pure, especially free of even trace contamination of non-specific proteases from the host organism. Since no protease will fit these requirements for all possible proteins of interest, industry will need a number of proteases to be developed for use in such a process. These proteases will preferably have a different specificity. A case-by-case screening can determine the optimal processing strategy for each polypeptide of interest.

This invention relates to the use of caspase activity to cut an artificial fusion protein, comprising a first part comprising a protein or peptide as a fusion part, a second part comprising a linker artificially designed to maximize cutting with the enzyme, and a third part comprising the protein or peptide of interest to be isolated in a mature and functional form. The caspase protein itself can be part of a fusion protein with the intention to e.g. optimize expression level or solubility of the caspase enzyme, or to optimize the easy recovery of the caspase enzyme, or to enable the caspase enzyme to be coupled efficiently to a solid support. The first steps in the elaboration of the processing consist of choosing a caspase, followed by designing, choosing or selecting a linker sequence that is cut by the caspase of choice.

Cloning of Caspases

Gene sequences of caspases genes can be found at the National Centre for Biotechnology Information (http://www.ncbi.nlm.nih.gov/). Caspases can be cloned from cDNA using appropriate primers hybridizing at the beginning and at the end of the coding sequence. Using the PCR reaction, an amplified DNA can be obtained and cloned in a suitable plasmid. Alternatively, the cDNA library is cloned and seeded to single colonies. The colony lysates can then be probed with a marked oligonucleotide that hybridizes specifically with the caspase gene. These techniques are known to a person skilled in the art.

Designing a Caspase Digestible Linker in a Fusion Protein

Preferably the linker sequence comprises a target sequence of a natural occurring substrate of the caspases. Most preferably, the linker comprises the most efficiently cut target sequence known or isolated for the caspase used. It is possible that this sequence is not a naturally occurring target sequence, but derived from a consensus sequence, or isolated by a screening method, or selected from a biological enrichment method, or obtained after a biological selection method.

Table I gives examples of target sequences obtained by comparing natural target sequences, or by synthesising sequences isolated by random library screening. The optimized sequences may also be obtained by comparing specific enzyme activity of the caspase on a peptide substrate.

Some preferred target sequences are:

| Caspase Group | Caspases | Preferred sequence |
|---|---|---|
| Group I | 1, 4, 5, 13 | WEHD (SEQ ID NO 11) |
| | | LEHD (SEQ ID NO 12) |
| Group II | 2, 3, 7, CED-3 | DEVD (SEQ ID NO 13) |
| | | DEHD (SEQ ID NO 14) |
| Group III | 6, 8, 9, 10, | VEHD (SEQ ID NO 15) |
| | | LEHD (SEQ ID NO 16) |

Preferably, the linker sequence further comprises a spacer region to increase the space between the fusion partner F and the caspase cutting site in the linker. Most preferably, the linker is designed or optimized in such a way, that the caspase target sequencers easily accessible by the caspase enzyme.

The linker sequence can be further optimized in order to increase the solubility of the fusion product. This can be done, as a non-limiting example, by enriching the sequence with hydrophilic amino acids, or with charged amino acids. The current state of the art makes it possible to design experiments in order to select linker sequences that would improve the solubility by screening or selecting a number of variants. These variants may be a defined number of options derived from a calculated choice, or derived from a randomized library of sequences.

The linker sequence can also be further optimized to comprise sequences that are recognized by a ligand or antibody in order to serve as an affinity target in an affinity based separation step, or to be used for easy and sensitive detection of the protein incorporating the sequence. Also, the linker may contain a high concentration of a certain class of amino acids, in order to improve adsorption to non-specific chromatography columns, such as ion exchange, hydrophobic interaction, ligand binding.

Construction of a Fusion Protein

The fusion construct comprises a combination of the general formula
F-L-G
where F is a fusion partner of choice, L is the linker sequence chosen, and G is the gene of interest, encoding the polypeptide of interest.

The fusion partner of choice can be chosen from public domain knowledge, on the basis of previous experience that fusing a gene G to the fusion partner F would improve the expression level and/or the solubility of the entire fusion gene. Examples of fusion partners that have been described for these purposes are:

The gluthation S-transferase gene GST, E. coli thioredoxin TRX, NusA, Chitin binding domain CBD, Chloramphenical acetyl transferase CAT, protein A (protA), LysRS, maltose binding protein (MBP), ubiquitin, calmodulin.

Other fusion partners can be chosen on the basis of rules that predict proteins to be effective as a fusion partner in order to obtain a higher expression and/or to improve the solubility of the fusion construct. U.S. Pat. No. 6,207,420 describes such a method based on the hydrophilicity index of the protein identified as a fusion partner.

The fusion partner can also be chosen to include a detection or affinity purification tag into the fusion protein. Of particular interest is a tag that allows for affinity purification of the fusion protein. These tags can be based on chemical principles, or are selected as epitopes for specific antibodies.

Examples of purification tags based on a strong selective chemical interaction to be used as a very selective purification step are a polyhistidine tag (H), that can be used in immobilized metal affinity chromatography, or a polyarginine tag that can be retained in high-salt ion exchange chromatography.

The fusion partner can be chosen from a combination of fusion proteins, a combination of tags or a combination of tags and fusion partners. For example, F could consist of a GST fusion protein F′ fused N-terminally to a HIS tag H, where
F-L-G=H-F′-L-G
or, in the case of a combination of fusion partners
F1-F2-L-G

In such a construct, affinity or detection peptides (tags t) can be placed in and between the fusion proteins or fusion protein domains Fi:
(t)-F1-(t)-F2-(t)-L-G

The N-terminus of the fusion protein comprising the F-L-G structure can be modified with a signal sequence. A signal sequence is a defined functional unit that allows the fusion protein to be secreted. In eukaryotic cells this will result in delivery of the fusion protein to the endoplasmatic reticulum, and possibly to the extra cellular medium. In Gram positive cells, this signal peptide guides the fusion protein through the cytoplasmic membrane into the medium. In Gram negative-cells the signal sequence guides the fusion protein to the periplasmic space between the cellular and the outer membrane, and occasionally the fusion protein is also released in the culture medium.

In the example where the fusion protein is secreted the formula for the gene construct would be
S-F-L-G
where S is a signal peptide and F is the fusion partner, there also may consist of a combination of sequences tₙ-Fᵢ where n and i are determined according to the application. Other fusion partners or tags can be inserted, either between the linker and the protein of interest or at the C-terminal of the protein of interest. However, these sequences will not be removed from the protein of interest after the linker sequence L is processed by a Caspase enzyme.

The fusion protein comprising the F-L-G parts is made by recombinant DNA techniques. The coding sequence of the complete fusion gene should preferably be determined before cloning. The coding sequences of the different parts in the fusion gene can be assembled using restriction enzymes, which cut specific sites in DNA molecules. Compatible sites can be religated again and this allows for constructing fusion genes. Alternatively, parts of the fusion gene or the complete fusion gene can be constructed by assembling pieces of synthetic DNA. Another possibility is to amplify the regions to be combined by the polymerase chain reaction (PCR) technology, where amplified DNA pieces can be assembled by including appropriate restriction enzymes and/or by including sufficient overlap in the DNA pieces and performing a splice overlap extension PCR reaction. Al of these techniques are known to the person skilled in the art and can be found in recent laboratory manuals covering recombinant DNA technology. Other variations and combinations of these techniques will also allow for obtaining the fusion gene.

Production of the Fusion Protein

The fusion gene needs to be engineered in such a way that it is translated into protein by the host organism. As a host organism, any living cell or organism applies. Living cells or organisms can be of prokaryotic or eukaryotic nature. Common cells that serve as hosts for expression of recombinant genes are for instance:

Escherichia coli, Bacillus species, Streptomyces species, Yeast strains such as Saccharomyces, Schizosacharomyces, Pichia or Hansenula strains, Insect cells, mammalian cell lines, plant cells. Expression hosts can also be at the level of a multicellular organism such as transgenic plants, sheep, goat, cow, chicken and rabbit, whereby the product can be isolated either from organs or from body fluids such as milk, blood or eggs.

Alternatively, the gene can be translated into protein using cell free translation systems, possibly coupled to an in vitro transcription system. These systems provide all steps necessary to obtain protein from DNA by supplying the necessary enzymes and substrates in an in vitro reaction. In principle, any living cell or organism can provide the necessary enzymes for this process and extraction protocols for obtaining such enzyme systems are known in the art. Common systems used for in vitro transcription/translation are extracts or lysates from reticolocytes, wheat germ or Escherichia coli.

Isolation of a Recombinant Fusion Protein that can be Maturated by Caspase

In a preferred embodiment, the fusion polypeptide will be isolated and purified before processing with the caspase protease. In this strategy, the physicochemical features of the fusion part can be used for uniform, streamlined and highly specific purification of the fusion protein. The characteristics of the fusion part towards adsorption chromatographic medium, or specific affinity purification methods should be considered. As non-limiting examples, peptide tags can be included that increase the binding to ion exchange columns (e.g. poly-arginine), hydrophobic interaction columns (e.g. polyphenylalanine), or immobilized metal chelating chromatography (e.g. polyhistidine). Other non-limiting examples are fusion protein or domains that have an affinity for a substrate or ligand (e.g. maltose binding protein MBP, gluthathion S transferase GST, protein A, biotinylated peptides or domains, chitin binding domains CBD). Further non-limiting examples are the use of fusion partners that stay soluble at higher temperature (e.g. thioredoxin), or will reversibly precipitate at certain conditions. A purification scheme based on the properties of the fusion partner will most probably be applicable to the complete fusion protein. A combination of such specific purification methods can be used if the fusion part is composed of different peptides, domains or proteins, or when it shows a different selective behaviour on different chromatography media.

In Vitro Cleavage Reaction

The in vitro cleavage of a recombinant fusion protein with a caspase should preferably be carried out under specific conditions. It is by no means stated that the invention is limited to these reactions conditions. The pH of the reaction is preferably controlled to be between 6 and 9, more preferably between 7 and 8. There is no need to ad salt to the reaction, but the cleavage reaction can tolerate up to 1 M NaCl. Preferably the NaCl concentration is not higher than 0.2 M. Some additives can enhance the cutting reaction of the caspases. Such additives are CHAPS 0.1%, sucrose 10% or mannitol 10%. Caspase enzymes are sensitive to oxidation of the cystein residue in the active centre. In order to avoid this, inclusion of antioxidantia (such as ascorbic acid), reductantia (such as β-mercaptoethanol, DTT, cysteines) and chelating salts to capture oxidation catalysing ions such as Zn⁺⁺ (such as EDTA).

On Column Cleavage

It is possible to reduce the number of steps needed in the maturation of the fusion protein, subsequent removal of the enzyme and removal of the fusion part cut from the fusion protein. If an affinity tag is incorporated in the fusion part, the same affinity tag can be fused, e.g. by recombinant DNA technology, to the caspase. Using this strategy, the fusion protein can be captured on a solid support (which can be a chromatographic column), and then incubated with a caspase that shows affinity for the same solid support. After an appropriate incubation time, the liquid phase of the reaction vessel will contain the protein of interest, while both the fusion part and the enzyme are adsorbed on the solid phase. Care should be taken to reduce the exposure of the caspase to oxidative conditions, since then the active site cystein will be oxidised and the reaction not reach is full potential. Reductantia (if possible), antioxidantia and chelating salts to capture oxidation catalysts can be included in the reaction. Examples of such conditions are 1-10 mM DTT or β-mercaptoethanol, or 1% ascorbic acid, or 1 mM EDTA.

In Vivo Cleavage

Cleavage of the fusion peptide can also be induced in vivo. Cleavage in the cell or the medium of the bioreactor has the advantage that no post-productional processing is needed. However, the advantage of a specific affinity purification based on the properties of the fusion part is lost in this case.

Two alternative strategies can be applied. First, the caspase may be induced at the same time as the fusion protein. This can be realized by constructing a transcriptional operon of the caspase enzyme and the fusion protein, or by constructing a translational fusion including the caspase in the fusion protein, or by cloning the caspase behind a separate promoter which is induced at the same time as the one in front of the fusion protein. The latter can be realized by using the same promoter in two transcriptional cassettes, or by using two promoters that are induced with the same inducer (e.g. IPTG/lactose), or by using two promoters, that are inducible with different agents, whereby both agents are added at the same time. Alternatively, the caspase enzyme can be induced at a different time point than the onset of production of the fusion protein. The caspase can be produced before or more preferably after the production onset of the fusion protein. In the latter case, the protein of interest will more likely fold to a soluble, active protein.

A first aspect of the invention is a method of producing a polypeptide of interest in a mature form, comprising

producing, in a suitable host, a fusion protein that comprises an amino terminal polypeptide, a linker comprising a caspase recognition site and a polypeptide of interest

cleaving said fusion protein by a caspase

isolating the mature polypeptide of interest.

In another aspect the invention relates to a method for producing a protein or polypeptide having a predetermined amino-terminal amino acid residue, comprising: a) expressing the protein or polypeptide in a host cell as a fusion protein wherein the amino terminus of the protein or polypeptide is fused to one or more fusion partners with a linker, the fusion protein being specifically cleavable by a caspase at the junction of the linker with the amino-terminal amino acid residue of the protein or polypeptide, the host cell lacking a caspase which cleaves the fusion protein at the junction of linker and the amino-terminal amino acid residue of the protein or polypeptide; b) isolating the fusion protein from the host cell; and c) contacting the fusion protein with an extract containing a caspase which specifically cleaves the fusion protein at the junction of the linker and the amino-terminal amino acid residue of the protein or polypeptide, said extract being derived from cells which produce said caspase by recombinant DNA methods.

The suitable host can be any host, and comprises both eukaryotic and prokaryotic host cells. As a non limiting example, the host can be a mammalian cell, and insect cell, a plant cell or a complete plant, a yeast cell, a fungal cell, a gram positive bacterium such as Bacillus sp or lactic acid bacteria, or a gram negative bacterium such as E. coli. Preferably said host is a prokaryotic cell, more preferably said host is a gram-negative bacterium, most preferably said host is Escherichia coli.

Preferably, said caspase recognition site comprises the amino acid sequence DXXD. More preferably, said caspase recognition site comprises either the sequence DEVD or DEHD.

Preferably, said linker sequence has been optimised for optimal caspase processing. Methods to optimise the linker sequence are known to the person skilled in the art, and include, as a non-limiting example, the insertion of a spacer region at the aminoterminal end of the caspase recognition site. The spacer region preferably comprises at least 1, more preferably at least 2, 3, 4, 5, 6, 7, 8, 9, 10, 11, 12, 13 or 14 additional amino acids, more preferably said spacer region comprises at least 15, 20 or 25 additional amino acids.

The caspase used can be any caspase. However, preferably said caspase is selected from the group consisting of caspase-2, caspase-3, caspase-7, CED-3, reversed caspase-3 and reversed caspase-7. More preferably, said caspase is caspase-3 or caspase-7. Even more preferably, said caspase is caspase-3.

In a preferred embodiment, the cleavage by the caspase is carried out in vitro, after isolating the fusion protein from the host cell, by mixing the fusion protein wit a caspase, preferably a purified caspase, in a suitable reaction mixture. However, the caspase cleavage may be carried out in vivo too, by inducing caspase activity in the same host cell as the one where the fusion protein is produced. The induction of the caspase activity may occur simultaneously with the production of the fusion protein, or the production of caspase and fusion protein may be sequential.

The caspase processing is interesting because of its high efficiency, even for mature proteins comprising an amino terminal amino acid that is difficult to process using other proteases. For normal amino terminal amino acids (i.e no P, D or E), preferably a yield of at least 80%, preferably 90% of mature polypeptide of interest is obtained within 60 minutes after initiation of the cleavage reaction. Cleavage of the fusion protein can be judged or examined by eye, ie on an SDS-Page gel, or can be measured by densitiometric scanning.

When a mature polypeptide with a proline as amino terminal amino acid is processed, preferably a yield of at least 20%, more preferably a yield of 25% of mature polypeptide is obtained. When a mature polypeptide with either a glutamine or an asparagine as amino terminal amino acid is processed, preferably a yield of at least 60%, more preferably a yield of 75% of mature polypeptide is obtained. Preferably said yields are obtained within 60 minutes after initiation of the cleavage reaction.

Definitions

The following definitions are set forth to illustrate and define the meaning and scope of various terms used to describe the invention herein.

The notation ‘DEVD/S’ (SEQ ID NO 17) means that a serine (S) follows the DEVD recognition sequence and is released after processing as the first amino acid of the mature protein.

The notation ‘DEVD/’ means that the polypeptide is cut after the last D irrespective of the first amino acid following the cutting site.

The terms ‘protein’ and ‘polypeptide’ as used in this application are interchangeable. ‘Polypeptide’ refers to a polymer of amino acids and does not refer to a specific length of the molecule. This term also includes post-translational modifications of the polypeptide, such as glycosylation, phosphorylation and acetylation.

The term ‘polypeptide of interest’ means the polypeptide that should be produced by the processing, and will be used for further applications. Preferably, it is a biological active molecule, such as an enzyme of a cytokine.

The ‘mature form’ or ‘mature polypeptide’ of interest refers to the polypeptide of interest in its biologically active form, without prepeptides or leader sequences. Mature form and mature polypeptide is used when we want to stress that the protein is starting with the amino terminal amino acid of the polypeptide of interest occurring under its biological active form. Preferably, said biological active form is identical to the mature form of the polypeptide of interest as it occurs in nature.

The ‘yield’ of the cleavage reaction is defined as the ratio of the protein of interest that is obtained by the cleavage reaction, on the theoretical amount of protein of interest that can be obtained, assuming that all fusion protein would be cleaved, expressed as a percentage.

### BRIEF DESCRIPTION OF FIGURES

FIG. 1: Caspase classification towards specificity. The figure shows the target sequence in the active site of the enzyme. The enzyme receiving pockets are denoted S1-S4, while the amino acid positions of the target sequence are denoted P1-P4. While Group II caspases prefers an aspartyl at position P4, Group I caspases prefer a hydrophobic residue at this position and Group III caspases prefer an aliphatic group at the P4 position.

FIG. 2: Design of a fusion protein

Different linker sequences (1 to 5, corresponding with SEQ ID NO. 1 to 10: even numbers amino acid sequences, uneven numbers nucleic acid sequences) were engineered between the fusion partner and the protein of interest. All constructs were equally expressed as a soluble protein. F: fusion partner, L: linker, P: polypeptide of interest.

FIG. 3 a) and b): Enterokinase can either fail to process an engineered linker, or degrade the target protein of interest. Both Caspase-3 and reverse Caspase-7 process the fusion protein with high efficiency in a very short time. EK: enterokinase recognition site; H: histidine tag; GST: glutathion S transferase; TRX: thioredoxin; F, L and P as in FIG. 2.

FIG. 4: Thrombin cutting of a GST-mLIF fusion protein needs a long processing time. This can be done more efficiently and in a very short time with Caspase-3. Two cutting sites were compared (DEVD/ and DEVD/S) who performed equally in the processing reaction. Abbreviations as in FIG. 3.

FIG. 5: The figure shows a titration of enzyme during 16 h incubation for Thrombin, and a 45 minutes incubation for Caspase-3. An amount of 50 ng is sufficient to process >90% of 10 microgram of a GST-mLIF fusion protein.

FIG. 6A: Influence of the P1′ site (first amino acid behind, i.e. downstream of, the cutting site) on cutting efficiency. The P1′ site was changed to all 20 possible amino acids and assayed for inhibition of processing.

FIGS. 6B and 6C: Influence of the linker sequence on the efficiency of cutting. Linker sequences consisting of a recognition site only are less efficient in cutting the fusion protein. In FIG. 6C it is shown that at least 2 μg of caspase 3 is needed (left construct) as compared to the control (right construct) where only 100 ng results in efficient cleavage of the fusion protein.

FIG. 7: Both mLIF and hIFN alpha were cut efficiently using Either Caspase-3 or reverse Caspase-7, with either the recognition site DEVD/ or DEVD/S.

FIG. 8: Screening of the amino acid sequence of mLIF, hIFN alpha, GST and TRX. Consensus sequences for Caspase group II recognition sites (DXXD) are boxed.

FIG. 9: Expression and purification of Caspase enzymes. Silver stain of Caspase-3 produced in Escherichia coli and purified as described.

FIG. 10: Co-expression of caspase with a fusion protein allows cleavage in vivo. A) plasmid maps for simultaneous or separate induction of caspase 3. B) Protein gel analysis of bacteria co-expressing caspase 3 with a cleavable fusion protein.

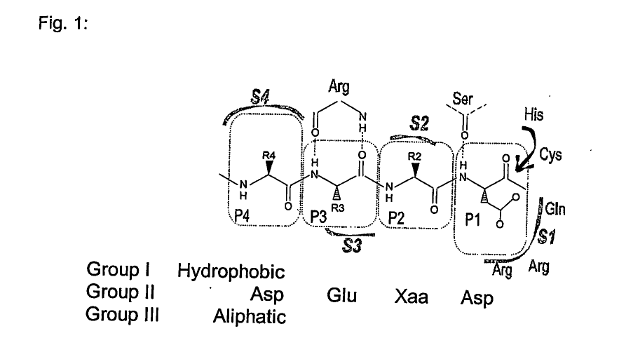

### EXAMPLES

### Example 1

### Designing a Cleavable Linker Sequence for Caspases Maturation

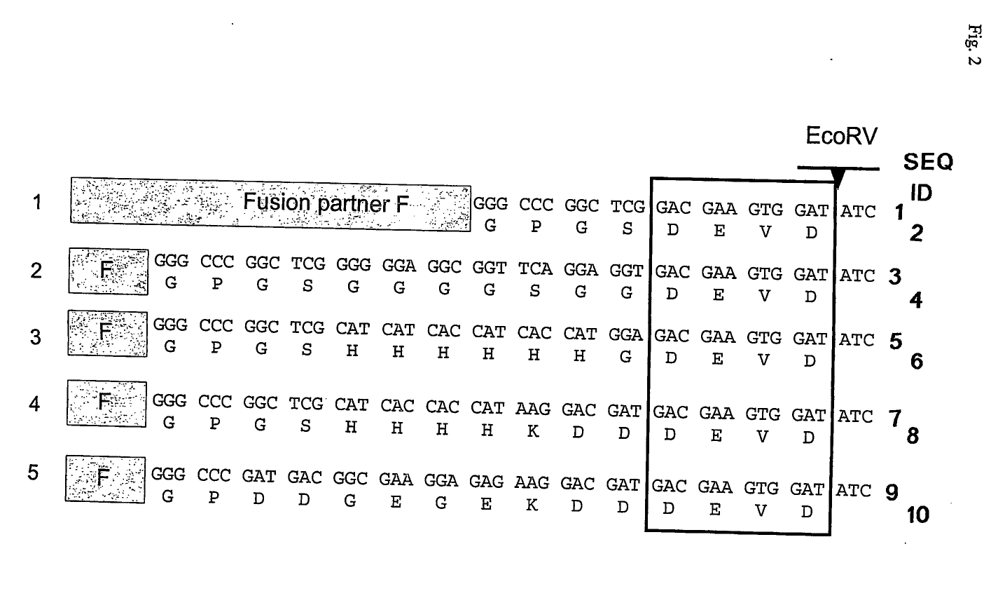

In order to use the method of the invention, one should first design an expression vector for a fusion protein construct, where the fusion construct is separated from the mature protein by a linker sequence. The linker sequence must contain a preferred recognition sequence for a Caspase protein. Examples of such recognition sequences are given in Table I. FIG. 2 shows some examples of linkers that were used for cleavage with Caspase 3.

### Example 2

### Cleavage with Caspase 3 Outperforms Cleavage with Industry Standard Proteases, which Lack Efficiency or Specificity

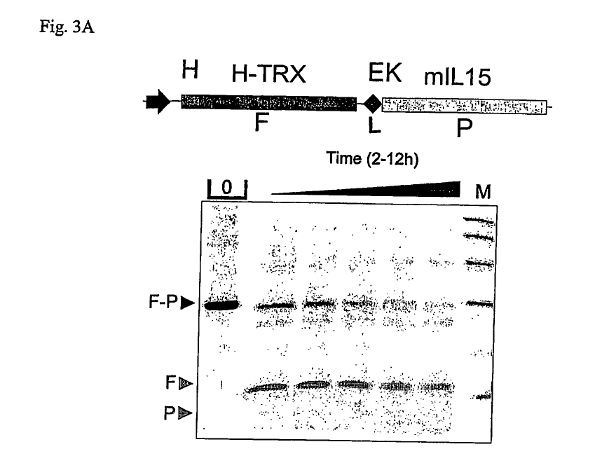

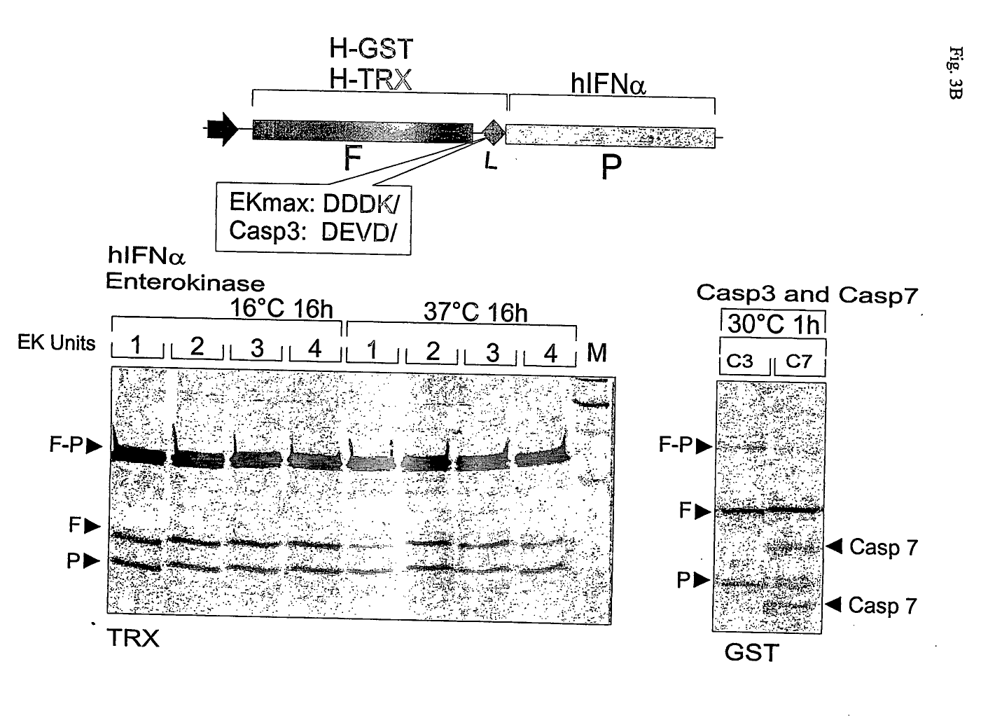

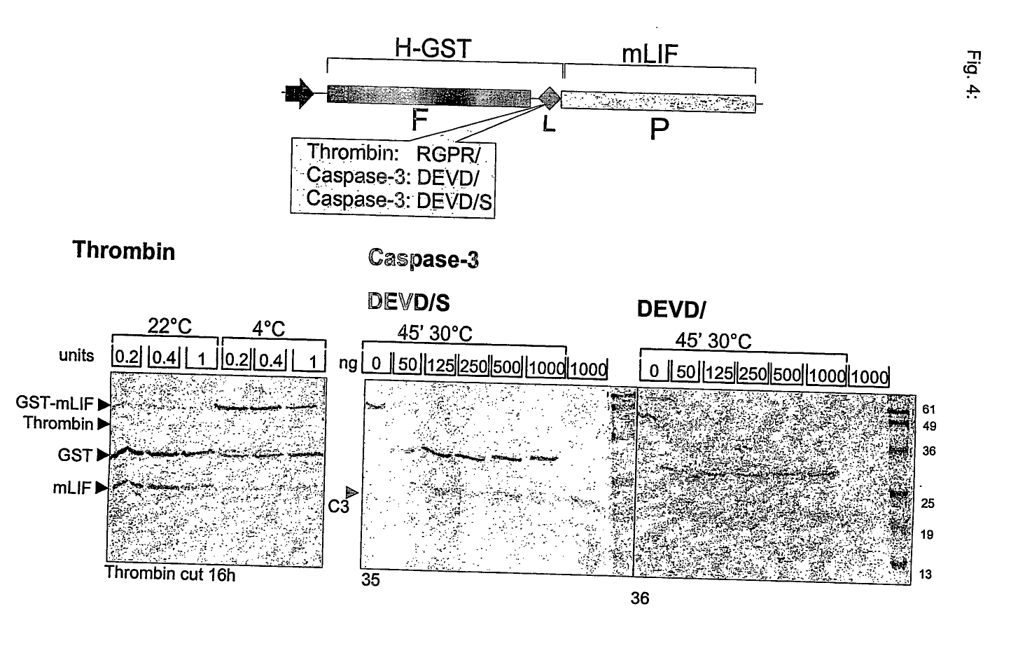

Industry standard enzymes for processing fusion proteins in order to obtain a mature protein without any additional amino acid, residual from designing the cleavage site, are not always efficient and can constitute the bottleneck in designing an efficient production process. FIG. 3 a) shows a cleavage experiment of a thioredoxin-murine interleukin 15 fusion gene, separated from each other with a recognition site for enterokinase (TRX-EK-mIL15). Cleavage with enterokinase required a long incubation time in order to process the molecule. In the end, the molecule was processed, which is apparent by the release of the thioredoxin (TRX) protein, but the murine interleukin 15 protein (mIL15) is also degraded by the enzyme or the enzyme preparation. In the other example, in FIG. 3 b), a fusion protein of TRX with human interferon alpha (hIFNα) comprising a linker sequence encoding a recognition site for Enterokinase (DDDK), TRX-EK-hIFNa, was compared to a fusion construct of gluthathion-S-tranferase (GST) with hIFNα comprising a Caspase-3 recognition site (DEVD) in the linker sequence, GST-C-hIFNα in the linker sequence. The fusion proteins were produced, purified, and processed with respectively Enterokinase and Caspase-3. 16 h incubation with Enterokinase resulted in only a minor amount of processing of the fusion protein. Caspase-3 on the other hand gave a high yield of processing even after only 45 minutes of incubation. Another industry standard for a protease to trim fusion proteins to obtain an unmodified mature protein sequence is Thrombin. FIG. 4 shows an example where murine LIF is fused to a GST fusion partner and a hexahistidine fusion partner (H), comprising either a thrombin protease recognition sequence in the connecting linker (H-GST-T-mLIF), or a Caspase-3 recognition sequence (H-GST-C-mLIF). Two different sites for Caspase-3 were introduced. Since it can be concluded from the analysis of the natural Caspase-3 sites that there is a clear preference for a small amino acid at the P1′ position, a linker sequence comprising a DEVD/recognition sequence where the mature mLIF sequence follows the sequence and is released after processing, was compared with a linker sequence comprising a DEVD/S, where a serine-mLIF follows the DEVD recognition sequence and is released after processing. The processing with Caspase-3 was far more efficient as compared with Thrombin, and the reaction was finished after 45 minutes when using Caspase-3 while up to 16 h were needed to process the H-GST-T-mLIF fusion protein with Thrombin. To our surprise, we could not see any difference in processing efficiency when using the H-GST-DEVD/mLIF or H-GST-DEVD/S-mLIF fusion proteins.

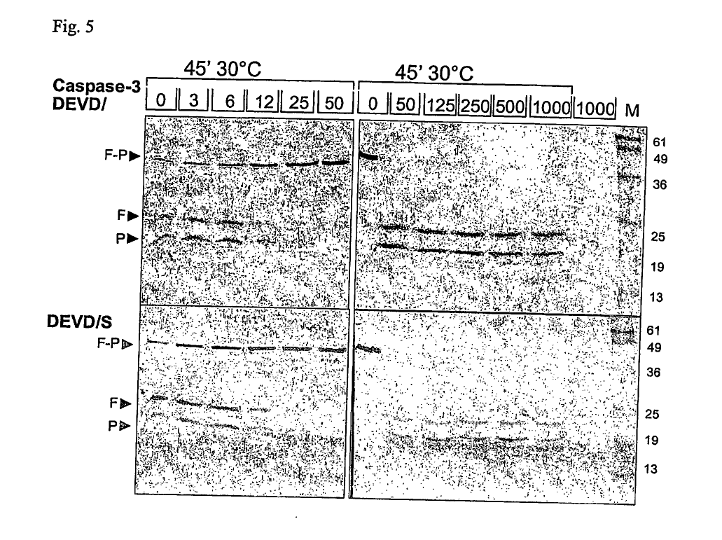

To test the efficiency of cuffing of such an engineered fusion protein, we titrated the enzyme on fusion protein. FIG. 5 shows an example of such a titration experiment on a H-GST-DEVD/mLIF and a H-GST-DEVD/S-mLIF. Again, no difference could be seen between both constructs, and the titration reaction shows that as little as 50 ng of enzyme is sufficient to process 10 microgram of protein.

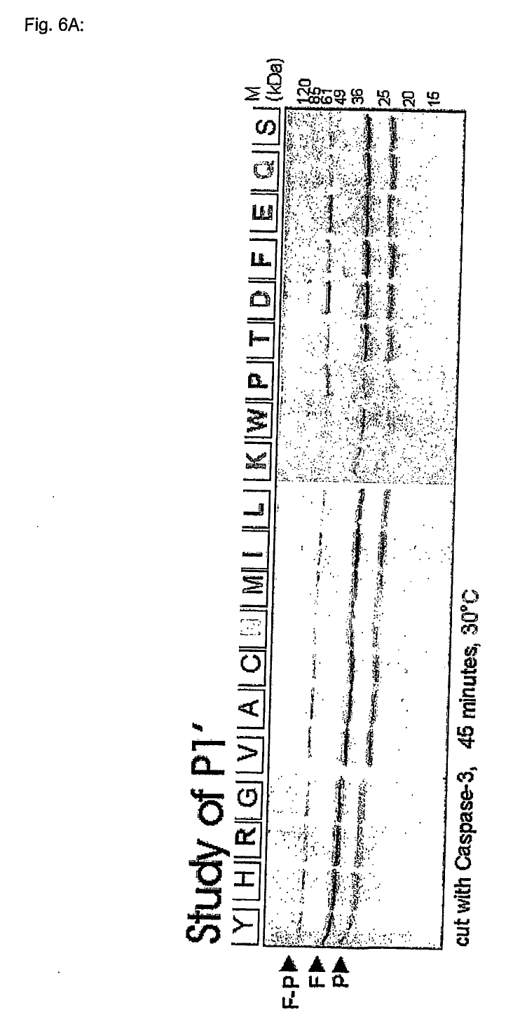

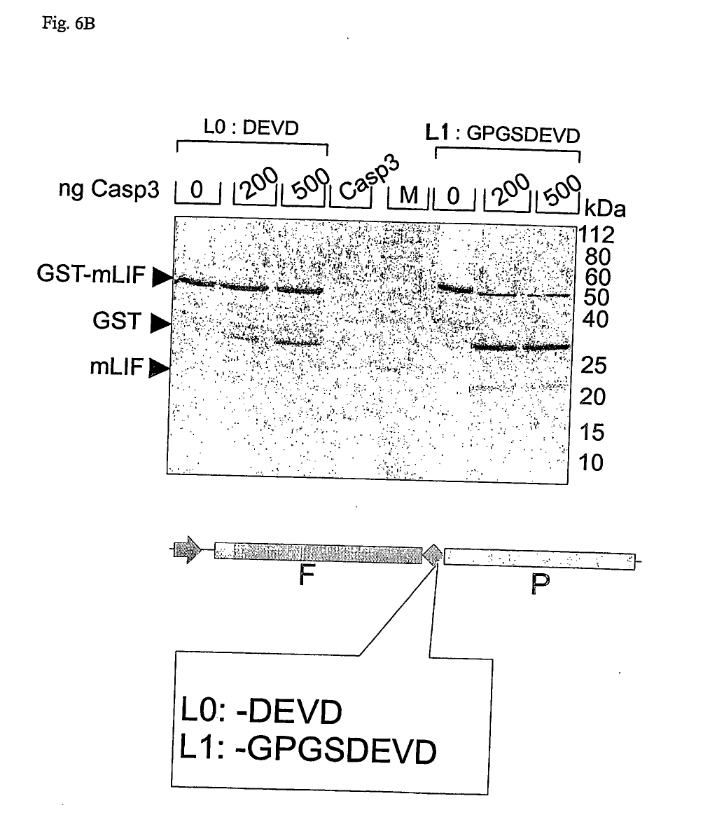

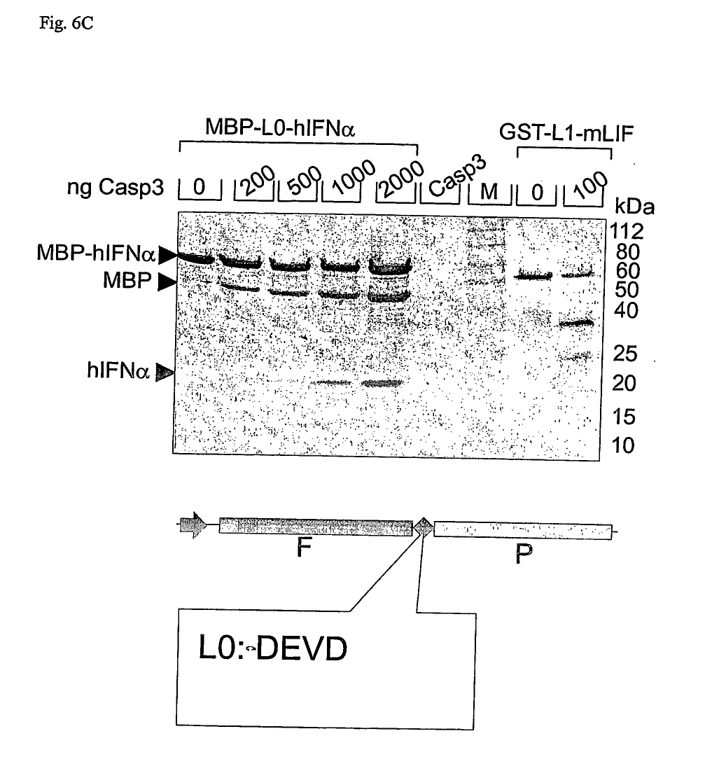

To test the limits of the Caspase-3 processing and the limitations in processing fusion proteins imposed by the P1′ sequence, we inserted 20 different amino acid at the P1′ position, preceding the mLIF sequence. All the 20 fusion proteins of the formula H-GST-DEVD/X-mLIF, where X is any of the possible 20 amino acids described by their single letter code, where expressed in E. coli, purified and used as substrate for cleavage with Caspase-3. FIG. 6 shows the results of the cutting experiment. To our great surprise, all amino acids in the P1′ position could be processed by Caspase-3. Proline (P) at position P1′ inhibited the processing about 75%, and the acidic amino acids glutamine (E) and asparagine (D) inhibited the processing about 25%, but no amino acid inhibited the Caspase processing 100%.

Next, we assessed the influence of the preceding amino acids by shortening and varying the linker sequences. Different lengths and sequences of the linker sequences before the DEVD-site had no influence on the cleavage reaction (FIG. 2). However, when no preceding linker sequence was present and the DEVD site followed the fusion protein directly, a marked decrease in the efficiency of cutting was noticed. FIG. 6B shows the fusion protein of GST and mLIF but now with only the DEVD site in the linker (L0: DEVD) compared to the construct where the DEVD site is preceded by a GPGS sequence (L1: GPGSDEVD). While 90% of the fusion product containing L1 was processed (as measured by densitometric scanning of the gel), only 10% processing yield was seen under these conditions when using the L0 linker. FIG. 6C shows a second example where 2 microgram of caspase 3 is needed to process only 50% of 10 microgram of MBP-L0-hIFNalpha. For 10 microgram of the control construct only 100 ng of caspase 3 results in efficient cleavage.

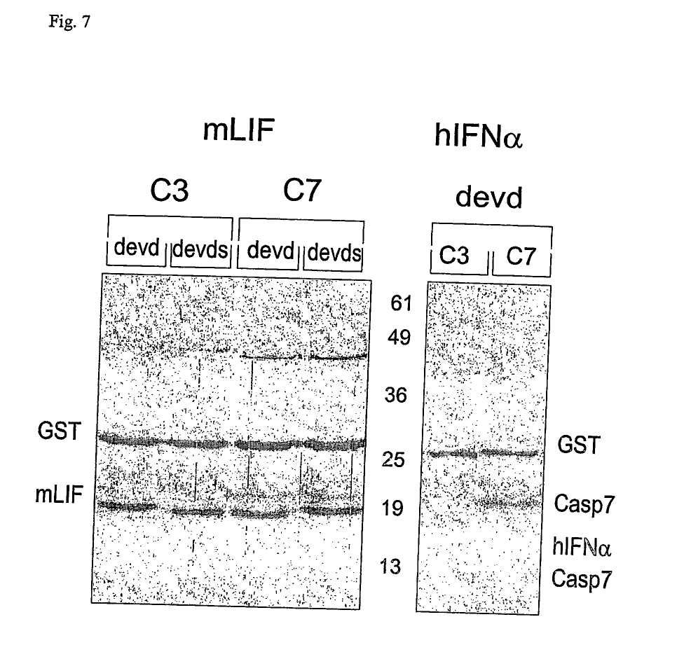

FIG. 7 shows that essentially the same results can be obtained by using caspase 7 or reversed caspase 7. In our hands, the murine caspase 7 was not as active as the murine caspase 3 and more enzyme needed to be added to obtain the same efficiency of cleavage.

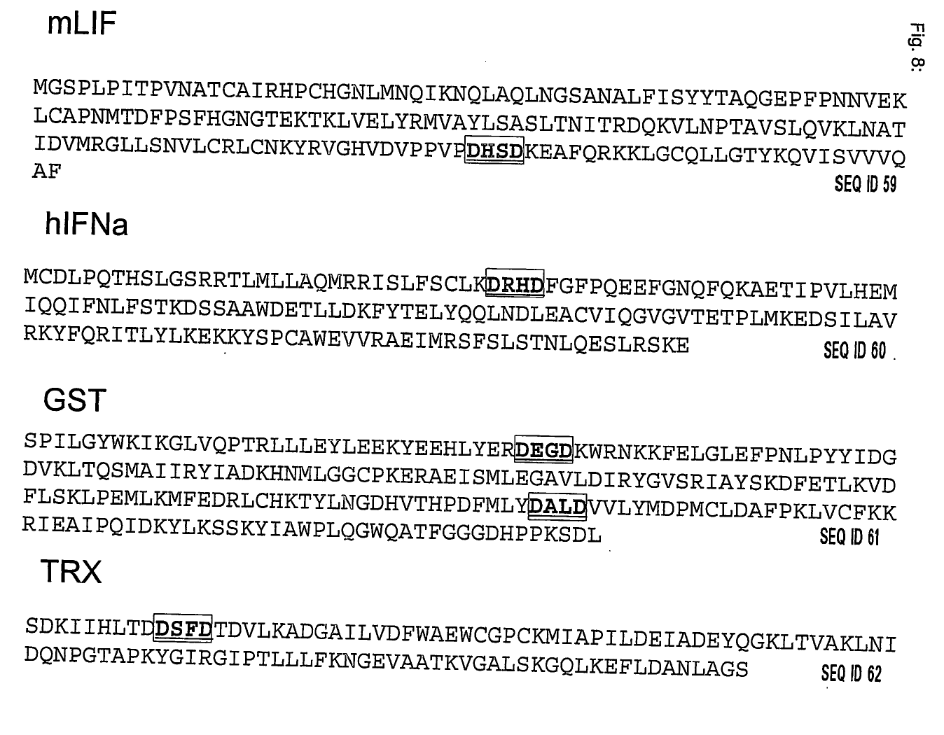

The specificity of the cutting reaction is illustrated by analysis of the sequence of the target proteins used in the example: both mLIF and hIFNa contain a consensus target sequence for group II caspases (DXXD/). Also the fusion partners used in these experiments contain the DXXD consensus sequence for group II caspase. Thioredoxin (TRX) contains a site, while the GST fusion partner even contains 2 target sequences (FIG. 8). One would expect further degradation of a fusion product comprising these proteins. To our great surprise, none of these sites was cut even upon prolonged incubation with Caspase-3 or Caspase-7, proving the specificity of the reaction towards the engineered sequence in the linker.

We conclude that the Caspases are a good choice to be used as a processing enzyme for fusion proteins since any amino acid can be tolerated at the P1′ position, they are efficient in cutting (usually complete after 45 minutes reaction time), and the sequence specificity is sufficient to prefer the processing at the engineered site, even if the target protein contains consensus recognition sequences for the Caspase enzymes.

### Example 3

### Expression and Purification of Caspases

The primary structure of the caspase zymogens or procaspases consists of a prodomain followed by a large subdomain of around 20 kDa (p20), and a smaller subdomain of around 10 kDa (p10). A combination of the p10 and p20 is denoted as p30. Active caspase can be obtained by co-expressing the processed subunits as separate subunits. In eukaryotic expression hosts this can be done by co-transfection of two plasmids containing both a promoter followed by a p10 or a p20 encoding gene. Alternatively, an internal ribosomal entry site can be used to express both subunits from a single transcript. In prokaryotes, also two promoter construct can be made, or the two genes can be placed in a single operon. The correct start position of both coding sequences should be engineered to optimize translation initiation in the host cell chosen, as known in the art.

Also, the caspase enzyme can be expressed as a p30, or as a procaspase. For most caspases, overexpression will induce self-activation by autoprocessing, resulting in the release of the p10 and the p20 from the p30 or the procaspase in vivo. The p10 and the p20 form a heterodimer that in turn will dimerise, so the final enzyme is a (p10-p20)₂ tetramer. Full length cDNA of caspase genes was used to clone the p30 caspases. Using synthetic oligonucleotide primers, appropriate restriction sites were engineered for subsequent cloning. The p30 caspase cDNA was clones behind the lambda PL promoter as described in Van de Craen et al. 1999, Cell Death and Differentiation a, 1117-1124, and induced by induction of an antirepressor as described in WO9848025. For convenience, the Caspase enzymes were tagged with a hexahistidine sequence at the N-terminus of the p20 sequence, or at the C-terminus of the p10 sequence, if the caspase is in the natural orientation. A reversed orientation can also be engineered as described in U.S. Pat. No. 6,379,950. In this case, the polyhistidine tag was either at the N-terminus of the p10, or at the C-terminus of the 20.

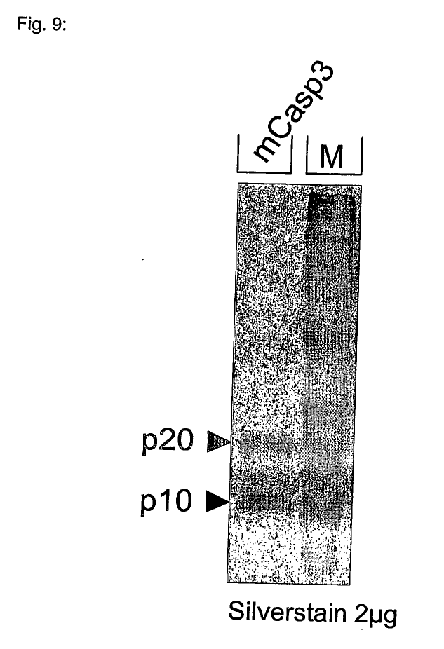

Upon induction of the Caspase gene in Escherichia coli according to WO9848025, the processed forms of the p10 and of the p20 were obtained. The bacterial pellet was resuspended in buffer A containing 20 mM Tris HCl pH 7.5; 10% glycerol; 1 mM oxidized gluthathione, 500 mM NaCl, 1 mM phenylmethylsulfonyl fluoride (PMSF), 50 mM leupeptine and 20 μg/ml aprotinin. The resuspended pellet was lysed by sonication or in a French Press. Insoluble proteins were removed by centrifugation or ultrafiltration. The cleared lysate of the bacterial culture was passed over a DEAE column to remove bacterial DNA. The flow-through was loaded on an immobilized metal chelating column containing 50 mM imidazol. Bound material was eluted from the column using 200 mM imidazol with 20 mM Tris-HCl pH 7.5, 10% glycerol, 1 mM oxidised gluthathione, 50 mM NaCl and dialysed to PBS buffer containing 1 mM oxidised gluthathione. The sample was applied over a mono-Q column (Amersham Bioscience) and eluted with a gradient of 0 to 1 M NaCl. FIG. 9 show a typical preparation of purified murine Caspase-3 from Escherichia coli.

Expression of the caspases could be increased by using fusion proteins. The caspases are fused to the C-terminus of a fusion partner such as thioredoxin, gluthathione S-transferase, maltose binding protein, NusA or others. The fusion between the fusion partner and the caspase gene can contain a protease site to release the caspase after production. Also, this protease site can be a target site for the Caspase itself, so the fusion protein is autoprocessed upon production of the active caspase.

### Example 4

### Caspases Maturation of Fusion Proteins In Vivo

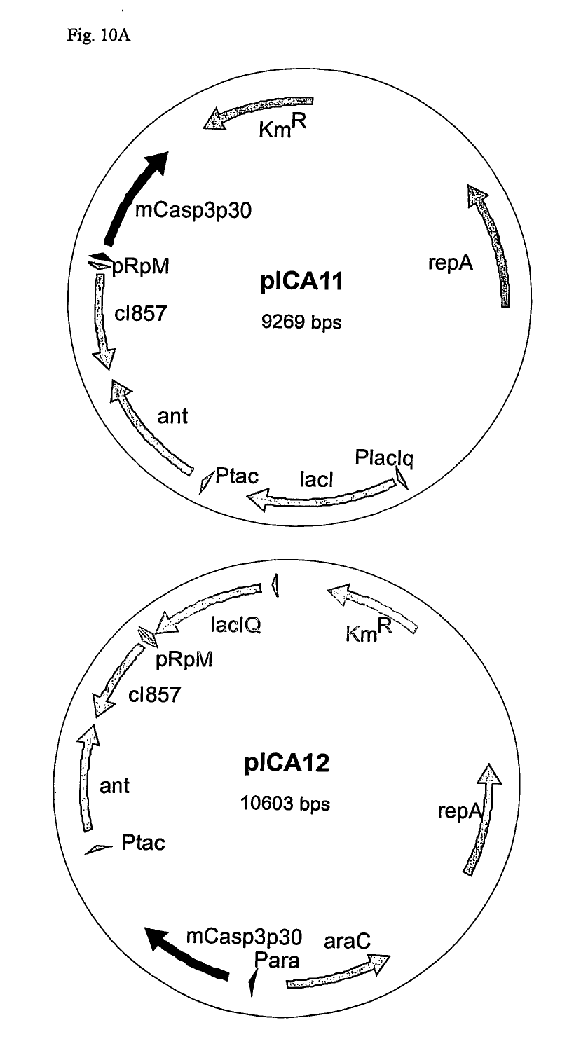

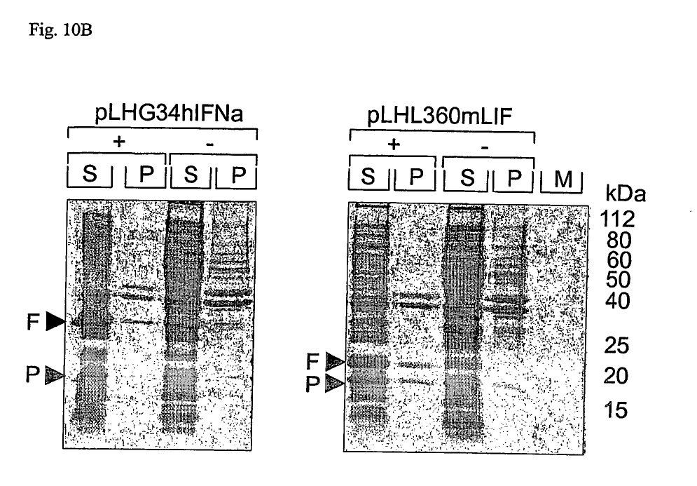

The cleavage reaction can be induced in vivo by cotransfecting the host cell with an expression vector for the Caspase enzyme. We have illustrated this by cloning the caspase gene behind the lambda PR promoter present on pICA2 plasmid that is described in WO9848025. A derivative of pICA2 had the promoter controlling ant (PN25/O2) changed to the Ptac promoter (pICA10). Next the gene for murine caspase 3 was cloned behind the PR promoter. The resulting pICA11 vector induces caspase together with the fusion protein (FIG. 10A). This results in the cleavage of a fusion protein comprising a DEVD in the linker sequence. FIG. 10B illustrates this using a GST-L1-IFNalpha fusion and a cleavable fusion protein containing mLIF. Quantitative cleavage occurred in vivo and no unprocessed fusion protein F-L1-P could be detected. Since in some cases the protein of interest needs a certain time to fold correctly, even when fused to the fusion partner, it is preferable that the induction of the Caspase enzyme will be independently regulated from the induction of the fusion protein. In this strategy, it is most preferable to first induce the fusion protein and after a time to be determined experimentally, induce the Caspase enzyme. Since the Caspase enzymes are active in the cytoplasm of E. coli and other host organisms, the fusion protein will be cut in vivo and the mature protein will be released. This strategy looses the advantage of a possibility for affinity purification, but avoids the process of in vitro cleavage and the consumption of purified Caspase enzyme.

A Caspase-3 p30 protein was cloned fused to a hexahistidine tag behind a Xanthosine promoter as described in PT102705. The H-GST-DEVD/mLIF fusion protein was induced using the lambda PL/ant induction system as described in WO9848025. After 4 h of induction by the addition of IPTG 1 mM, 0.2% of xanthosine was added to the medium. The Caspase-3 activity is induced and processes the fusion protein in vivo. In another strategy, the caspase 3 gene was cloned behind the arabinose promoter and inserted in pICA10. The resulting pICA12 (FIG. 10A) induces caspase upon the addition of 0.1% arabinose, while the fusion protein will be induced by the addition of lactose or IPTG.

### Example 5

### A Caspases Maturation Bioreactor

In order to take advantage of the possibility to use the fusion construct for affinity purification, but to minimize the consumption of purified enzyme, a bioprocess reactor can be devised based on Caspase enzyme coupled to a solid support. In this way, easy recuperation can be achieved and the enzyme reactor can be reused several times. Also, the removal of the enzyme is not necessary anymore since the enzyme remains bound to the solid phase support and thus does not contaminate the protein of interest, which is in the liquid phase. Caspase—was purified and coupled to a preactivated NHS column (Amersham Bioscience) according the supplier's instructions. Non-bound enzyme was removed by several washes. A purified fraction of GST-L-mLIF was loaded into the reactor, and incubated for 2 hours. The reactor was flushed and the eluted protein captured on an immobilized metal affinity column. The flow-through contains the processed protein of interest while the fusion part is captured on the IMAC column. The Caspase-3 reactor could be used more than once.

TABLE I
All proteins are referenced in Earnshaw, Martins and Kaufmann,
Mammalian caspases: structure, activation, substrates and functions
during apoptosis. Annu. Rev. Biochem; 1999, 68: 383-424.
Naturally occurring target sites for Group II caspases (2, 3, 7)

| | | |
|---|---|---|
| Protein | P4-P1 | P1′ |

Consensus

| | | |
|---|---|---|
| PARP | DEVD | G |
| RepC LS | DEVD | G |
| BetaII- | DEVD | S |
| spectrin | | |
| Fodrin (a) | DETD | S |
| | (18*) | |
| Mst1 K | DEMD | S |
| | (19) | |
| Mst2 K | DELD | S |
| | (20*) | |
| D4 GDP DI | DELD | S |
| SREBP | DEPD | S |
| | (21*) | |
| Rb | DEAD | G |
| | (22*) | |
| PP2A Aalpha | DEQD | S |
| | (23*) | |
| ICAD | DEPD | S |
| PPLA2 | DELD | A |

Larger P1′

| | | |
|---|---|---|
| DNA-P-Kos | DEVD | N |
| | (24*) | |
| PKCteta | DEVD | K |

Lacking P3 Glu

| | | |
|---|---|---|
| MEKK-1 | DTVD | G |
| FAK (a) | DQTD | S |
| | (25*) | |
| PKSrelK2 | DITD | C |
| | (26*) | |
| PKCdelta | DMQD | M |
| | (27*) | |
| Fodrin (a) | DSLD | S |
| | (28*) | |
| 70kDaU1snRNP | DGPD | G |
| | (29*) | |
| mdm2 | DVPD | C |
| | (30*) | |
| IkappaB | DRHD | A |
| | (31*) | |
| Waf1 | DHVD | L |
| | (32*) | |
| Kip1 | DPSD | S |
| | (33*) | |
| Huntington | DSVD | L |
| | (34*) | |
| DPAP | DSLD | S |
| | (35*) | |
| Presenilin-2 | DSYD | S |
| | (36*) | |
| Gelsolin | DQTD | G |
| | (37*) | |
| Topol | DDVD | Y |
| | (38*) | |
| Bcl-2 | DAGD | V |
| | (39*) | |
| RAS-GAP | DTVD | G |

Lacking P4 Asp

| | | |
|---|---|---|
| Stat1 | MELD | A |
| | (40*) | |
| Cytokeratin | VEVD | A |
| 18 | (41*) | |
| Lamin B1 | VEVD | S |
| FLIPL | LEVD | G |
| | (42*) | |

Lacking P3 Glu + P4 Asp

| | | |
|---|---|---|
| Bid | LQTD | G |
| | (43*) | |
| FAK (a) | VSWD | S |
| | (44*) | |
| PITSLRE 2-1 | YVPD | S |
| | (45*) | |
| p21 K2 | SHVD | G |
| | (46*) | |
| Bcl-X | HLAD | S |
| | (47*) | |
| Presenilin-1 | ARQD | S |
| | (48*) | |
| Ca PK IV | PAPD | A |
| | (49*) | |
| pro-IL-6 | SSTD | S |
| | (50*) | |
| PKN | LGTD | S |
| | (51*) | |
| DCC | LSVD | R |
| | (52*) | |
| Calpastatin | ALDD | S |
| (a) | (53*) | |
| Calpastatin | LSSD | F |
| (b) | (54*) | |
| Calpastatin | ALAD | S |
| (c) | (55*) | |
| Bax | FIQD | R |
| | (56*) | |

*The numbers in brackets refer to the corresponding SEQ ID NOs.

### REFERENCES

Colussi, P. A., N. L. Harvey, L. M. Shearwin-Whyatt and S. Kumar (1998). “Conversion of procaspase-3 to an autoactivating caspase by fusion to the caspase-2 prodomain.” J Biol Chem 273(41): 26566-70.
Garcia-Calvo, M., E. P. Peterson, D. M. Rasper, J. P. Vaillancourt, R. Zamboni, D. W. Nicholson and N. A. Thornberry (1999). “Purification and catalytic properties of human caspase family members.” Cell Death Differ 6(4): 362-9.
Nicholson, D. W. (1999). “Caspase structure, proteolytic substrates, and function during apoptotic cell death.” Cell Death Differ 6(11): 102842.
Nicholson, D. W. and N. A. Thornberry (1997). “Caspases: killer proteases.” Trends Biochem Sci 22(8): 299-306.
Stennicke, H. R., M. Renatus, M. Meldal and G. S. Salvesen (2000). “Internally quenched fluorescent peptide substrates disclose the subsite preferences of human caspases 1, 3, 6, 7 and 8.” Biochem J 350 Pt 2: 563-8.
Stennicke, H. R. and G. S. Salvesen (1997). “Biochemical characteristics of caspases-3, -6, -7, and -8.” J Biol Chem 272(41): 25719-23.
Stennicke, H. R. and G. S. Salvesen (1999). “Catalytic properties of the caspases.” Cell Death Differ 6(11):1054-9.
Thornberry, N. A., K. T. Chapman and D. W. Nicholson (2000). “Determination of caspase specificities using a peptide combinatorial library.” Methods Enzymol 322: 100-10.
Van de Craen, M., W. Declercq, I. Van den brande, W. Fiers and P. Vandenabeele (1999). “The proteolytic procaspase activation network: an in vitro analysis.” Cell Death Differ 6(11): 1117-24.
Alnemri, E. (1999). Recombinant, active caspases and uses thereof. U.S. Pat. No. 6,379,950,
McCoy, J. and E. Lavallie (1993). Peptide and protein fusions to thioredoxin and thioredoxin-like molecules. WO9213955.
Smith, D. (1993). Tripartide fusion proteins of gluthathione S-transferase. WO9319091
Smith, D. (1988). Novel fusion proteins containing gluthathione S-transferase. WO8809372
LaVallie, E. (1994). Cloning of Enterokinase and method of use. U.S. Pat. No. 5,665,566,
Johnston, S. and W. Dougherty (1993). Method of isolation and purification of fusion polypeptides. U.S. Pat. No. 5,532,142

1. A method for producing a polypeptide of interest in a biologically active form, comprising
producing, in a suitable host, a fusion protein comprising an aminoterminal polypeptide, a linker sequence comprising a caspase recognition site and the polypeptide of interest,
cleaving said fusion protein by a caspase which specifically cleaves the fusion protein at the junction of the linker sequence and the amino-terminal amino acid residue of the mature polypeptide of interest, and
isolating the biologically active polypeptide of interest.

2. The method according to claim 1 wherein said suitable host is a prokaryote.

3. The method according to claim 2 wherein said prokaryote is Escherichia coli.

4. The method according to claim 1, wherein said caspase recognition site comprises the amino acid sequence DXXD, DEVD or DEHD.

5. The method according to claim 1, wherein said caspase is selected from the group consisting of caspase-2, caspase-3, caspase-7, CED-3, reversed caspase-3 and reversed caspase-7.

6. The method according to claim 1, wherein said linker sequence comprises at least 5 amino acids.

7. The method according to claim 1, wherein said linker sequence consists of the amino acid sequence DEVD or DEHD.

8. The method according to claim 1, wherein said linker sequence is represented by any of SEQ ID NOs 2, 4, 6, 8 or 10.

9. The method according to claim 1, wherein said cleaving of the fusion protein by the caspase is carried out in vitro.

10. The method according to claim 1, wherein a yield of at least 80% of mature polypeptide of interest is obtained within 60 minutes as judged by densitiometric scanning.

11. The method according to claim 1, wherein the caspase protein is produced in the same host as the fusion protein.

12. The method according to claim 1, wherein the caspase protein sequence is comprised in the fusion protein.

13. The method according to claim 11, wherein the production of the caspase protein and the fusion protein is sequential.

14. The method according to claim 13, wherein the caspase protein is produced after the fusion protein.

15. The method according to claim 10, wherein said cleaving of the fusion protein is carried out in vivo.

16. A method for producing a polypeptide of interest in a biologically active form according to claim 1, comprising:
producing in E. coli, a fusion protein comprising an aminoterminal polypeptide, a linker sequence comprising a caspase recognition site and the polypeptide of interest, wherein the linker sequence is represented by any of SEQ ID NOs 2, 4, 6, 8 or 10,
cleaving said fusion protein by a caspase which specifically cleaves the fusion protein at the junction of the linker sequence and the amino-terminal amino acid residue of the mature polypeptide of interest, and
isolating the biologically active polypeptide of interest.

17. A method for producing a polypeptide of interest in a biologically active form according to claim 11, comprising:
producing in E. coli, (1) a fusion protein comprising an aminoterminal polypeptide, a linker sequence comprising a caspase recognition site, the polypeptide of interest and a (2) a caspase protein, wherein said fusion protein and said caspase polypeptide are under the control of a different inducible promoter,
inducing expression of the fusion protein,
inducing expression of the caspase protein,
cleaving said fusion protein in vivo by the caspase protein which cleaves the fusion protein at the junction of the linker sequence and the amino-terminal amino acid residue of the mature polypeptide of interest, and
isolating the biologically active polypeptide of interest.

18. The method according to claim 17 wherein the amino acid sequence of the linker is represented by any of SEQ ID NOs 2, 4, 6, 8 or 10.

19. The method according to claim 16, wherein said caspase protein is caspase-3 or caspase-7.

20. The method according to claim 17, wherein a single polynucleic acid encodes the fusion protein and the caspase.

21. The method according to claim 17, wherein separate polynucleic acids encode the fusion protein and the caspase.

22. A polynucleic acid encoding the fusion protein as defined in claim 1.

23. A polynucleic acid encoding a fusion protein comprising an aminoterminal polypeptide, a mature polypeptide of interest and a linker sequence between the aminoterminal polypeptide and the mature polypeptide of interest, said linker sequence comprising a caspase recognition site at the junction between the linker and the amino terminal amino acid of the mature polypeptide of interest.

24. A polynucleic acid according to claim 23 further encoding a caspase protein.

25. A polynucleic acid according to claim 24 wherein said caspase protein is selected from the group consisting of caspase-2, caspase-3, caspase-7, CED-3, reversed caspase-3 and reversed caspase-7.

26. A polynucleic acid according to claim 22, wherein said aminoterminal polypeptide is selected from the group consisting of glutathione S-transferase gene GST, E. coli thioredoxin TRX, NusA, chitin binding domain CBD, chloramphenicol acetyl transferase CAT, protein A (prot A), LysRS, maltose binding protein (MBP), ubiquitin, calmodulin, DsbA, DsbC and lambda gpV.

27. A polynucleic acid according to claim 22, wherein said linker sequence comprises at least 5 amino acids.

28. A polynucleic acid according to claim 22, wherein said caspase recognition site consists of the amino acid sequence DXXD, DEVD or DEHD.

29. A polynucleic acid according to claim 22, comprising a linker sequence encoding the peptide represented in any of SEQ ID NOs 2, 4, 6, 8 or 10.

30. A polynucleic acid according to claim 22, wherein the expression of said fusion protein and/or said caspase protein is under the control of a suitable promoter.

31. A polynucleic acid according to claim 30 wherein said promoter is an inducible promoter.

32. A polynucleic acid according to claim 31 wherein the fusion protein and the caspase protein are under the control of a different inducible promoter.

33. A polynucleic acid according to claim 22, wherein said fusion protein further comprises a detection or affinity tag.

34. A polynucleic acid according to claim 33 wherein said detection or affinity tag is selected from the group consisting of a polyhistidine tag, a polyarginine tag, a streptavidin binding tag, a biotinylated tag and a tag recognized by an antibody.

35. A vector comprising the polynucleic acid of claim 22.

36. A host cell comprising the vector of claim 35.

37. A fusion protein encoded by the nucleic acid of claim 22.

38. A method of producing a polypeptide of interest in a mature and/or biologically active form which comprises transforming a host cell with the vector of claim 35.

39. A bioreactor suitable for the production of mature proteins of interest comprising:
(a) a fusion protein encoded by a polynucleic acid of claim 33, said fusion protein comprising an affinity tag having a specific affinity for a substrate, and
(b) a substrate,
wherein the affinity tag portion of the fusion protein is bound to the substrate.

40. A bioreactor suitable for the production of mature proteins of interest comprising:
(a) a fusion protein encoded by a polynucleic acid of claim 33, said fusion protein comprising an affinity tag having a specific affinity for a substrate, and
(b) a caspase enzyme coupled to a solid support,
wherein the caspase enzyme specifically cleaves the caspase recognition site in the fusion protein.

41. A method for producing a protein of interest comprising:
(a) producing a fusion protein according to claim 33, said fusion protein comprising an affinity tag or an aminoterminal polypeptide having a specific affinity for a substrate,
(b) introducing said fusion protein into a bioreactor comprising a caspase enzyme coupled to a solid support,
(c) incubating said fusion protein with the caspase enzyme for at least one hour, and
(d) washing the reactor and capturing the eluted fusion protein by binding to the substrate.
